SEQUENCE LISTING (23 sequences)

SEQ ID NO: 1 - DNA, 21 bp, Artificial Sequence
  Synthetic construct
gtgtggaaaatctctagcagt

SEQ ID NO: 2 - DNA, 21 bp, Artificial Sequence
  Synthetic construct
actgctagagattttccacac

SEQ ID NO: 3 - DNA, 19 bp, Artificial Sequence
  Synthetic construct
gtgtggaaaatctctagca

SEQ ID NO: 4 - DNA, 19 bp, Artificial Sequence
  Synthetic construct
actgctagatcggaagtgc

SEQ ID NO: 5 - DNA, 6 bp, Artificial Sequence
  Synthetic construct
actgct

SEQ ID NO: 6 - DNA, 79 bp, Artificial Sequence
  Synthetic construct
aatgatacggcgaccaccgagatctacactctttccctacacgacgctcttccgatctgtgtggaaaatctctagcagt

SEQ ID NO: 7 - DNA, 85 bp, Artificial Sequence
  Synthetic construct
caagcagaagacggcatacgagatcgtgatgtgactggagttcagacgtgtgctcttccgatctgtgtggaaaatctctagcagt

SEQ ID NO: 8 - DNA, 64 bp, Artificial Sequence
  Synthetic construct
aatgatacggcgaccaccgagatctacactctttccctacacgacgctcttccgatctagcagt

SEQ ID NO: 9 - DNA, 70 bp, Artificial Sequence
  Synthetic construct
caagcagaagacggcatacgagatcgtgatgtgactggagttcagacgtgtgctcttccgatctagcagt

SEQ ID NO: 10 - DNA, 76 bp, Artificial Sequence
  Synthetic construct
tgtgtgcccgtctgttgtgtgactctggtaactagagatcctcagacctttttggtagtgtggaaaatctctagca

SEQ ID NO: 11 - DNA, 78 bp, Artificial Sequence
  Synthetic construct
actgctagagattttccacactaccaaaaaggtctgaggatctctagttaccagagtcacacaacagacgggcacaca

SEQ ID NO: 12 - DNA, 79 bp, Artificial Sequence
  Synthetic construct
actggaagggttaatttactccaagcaaaggcaagatatccttgatttgtgggtctataacacacaaggctacttccca

SEQ ID NO: 13 - DNA, 79 bp, Artificial Sequence
  Synthetic construct
tgggaagtagccttgtgtgttatagacccacaaatcaaggatatcttgcctttgcttggagtaaattaacccttccagt

SEQ ID NO: 14 - DNA, 30 bp, Artificial Sequence
  Synthetic construct
cgataggatccgagtgaattagcccttcca

SEQ ID NO: 15 - DNA, 32 bp, Artificial Sequence
  Synthetic construct
actggaagggctaattcactcggatcctatcg

SEQ ID NO: 16 - protein, 171 aa, Artificial Sequence
  HIV-1-derived Integrase (QHIN 1)
MKHHHHHHMHGQVDCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNKKRKGGIGGYSAGERIVDIIATDIQTKE

SEQ ID NO: 17 - DNA, 20 bp, Artificial Sequence
  Synthetic construct
aatgatacggcgaccaccga

SEQ ID NO: 18 - DNA, 21 bp, Artificial Sequence
  Synthetic construct
caagcagaagacggcatacga

SEQ ID NO: 19 - DNA, 19 bp, Artificial Sequence
  Synthetic construct
gtgtgcccgtctgttgtgt

SEQ ID NO: 20 - DNA, 22 bp, Artificial Sequence
  Synthetic construct
ccacactaccaaaaaggtctga

SEQ ID NO: 21 - DNA, 20 bp, Artificial Sequence
  Synthetic construct
actccaagcaaaggcaagat

SEQ ID NO: 22 - DNA, 21 bp, Artificial Sequence
  Synthetic construct
tgggaagtagccttgtgtgtt

SEQ ID NO: 23 - DNA, 22 bp, Artificial Sequence
  Synthetic construct
aggatccgagtgaattagccct

(19) United States
(12) Patent Application Publication
(10) Pub. No.: US 2019/0194718 A1
(43) Pub. Date: Jun. 27, 2019

(54) GENERATION OF TAGGED DNA FRAGMENTS

(71) Applicant: QIAGEN GMBH, Hilden (DE)

(72) Inventors: Ioanna Andreou, Koeln (DE); Nan Fang, Neuss (DE); Dirk Loeffert, Hilden (DE); Annika Piotrowski, Hattingen (DE)

(21) Appl. No.: 15/110,470

(22) PCT Filed: Dec. 11, 2014
(86) PCT No.: PCT/EP2014/077306

§ 371 Date: Jul. 8, 2016

(30) Foreign Application Priority Data
Jan. 14, 2014 (EP) 14151017.2

(51) Int. Cl. C12Q 1/6806; C12Q 1/686; C12Q 1/6853; C12Q 1/6876

(52) U.S. Cl. CPC C12Q 1/6806; C12Q 1/686; C40B 30/00; C12Q 1/6876; C12Q 1/6853

(57) ABSTRACT

The present invention is directed to novel methods, kits and uses to be employed for the generation of tagged DNA fragments of a target DNA and nucleic acid molecules associated therewith

### CROSS-REFERENCE TO RELATED APPLICATIONS

This application is a U.S. national phase application of International Application No. PCT/EP2014/077306, filed Dec. 11, 2014, and which claims the benefit of priority of EP Application No. 14151017.2, filed Jan. 14, 2014. The content of these earlier filed applications are hereby incorporated by reference herein in thier entirety.

### SEQUENCE LISTING

The present application contains a Sequence Listing that has been submitted ASCII format via EFS-Web on October 23, 2018, containing the file name “17104_0058U1_Revised_Sequence_Listing.txt,” which is 8,192 bytes in size, created on Oct. 23, 2018, and is hereby incorporated by reference pursuant to 37 C.F.R. § 1.52(e)(5).

The present invention is directed to novel methods, kits and uses to be employed for the generation of tagged DNA fragments of a target DNA and nucleic acid molecules associated therewith.

### FIELD OF THE INVENTION

The present invention relates to the field of molecular biology, more particularly to the generation of DNA fragments and, specifically, to the generation of a plurality or library of tagged DNA fragments of a target DNA, respectively.

### BACKGROUND OF THE INVENTION

Tagged DNA fragments are required for many applications in modern molecular biology techniques. For example, in applications like next generation sequencing (NGS) the DNA to be sequenced has to be provided in fragmented form before amplification of the clusters which are finally the substrate for the sequencing reaction. In addition, adapter sequences have to be added to both ends of the template to ensure indexing, amplification of fragments and provision of a sequence that is specific for the sequencing primers.

Currently, differently methods are used to process the template and generate tagged DNA fragments or libraries thereof, respectively. Such known methods are based on physical or enzymatic digestion of nucleic acids and subsequently enzymatic reactions to prepare suitable ends for the sequencing, such as enzymatic end repair of the fragments, A-addition and adaptor legation.

In the art a method for preparing libraries of tagged DNA fragments is described comprising the enzymatic fragmentation and adaptor ligation in one step. Both reactions are performed by enzymes called transposases which use small dsDNA transposon-like molecules to simultaneously fragment and tag the template or target DNA. The fragmentation with simultaneous adaptor ligation is based on the use of transposases. This technology is disclosed in WO 2010/048605 A1. It is also the subject of Illumina's® Nextera™ DNA Kit.

However, the fragmentation with simultaneous adaptor ligation using transposases and dsDNA transposon-like molecules has several drawbacks. The cut-and-paste mechanism underlying the strand transfer reaction is complex. The transposase reaction can result in a change of the nucleotide sequences of both the dsDNA transposon-like molecules and the target DNA. A subsequent DNA sequencing reaction might then produce incorrect results. Furthermore, relatively long recognition sequences for the transposase need to be included into the dsDNA transposon-like molecules. These sequences will either be designed as part of the sequencing primer and cause less flexibility in working with different platforms and/or library indices, or sequenced as part of each sequencing template, causing waste of the sequencing capacity.

Against this background, it is an object of the present invention to provide for a method for generating tagged DNA fragments of a target DNA where the problems associated with the prior art methods can be reduced or avoided.

The present invention satisfies these and other needs.

### SUMMARY OF THE INVENTION

The present invention provides a method for generating tagged DNA fragments of a target DNA, comprising
(i) contacting said target DNA with an integrase and at least one DNA adaptor molecule that comprises an integrase recognition site, to obtain a reaction mixture,
(ii) incubating said reaction mixture under conditions wherein a 3′ processing of said at least one adaptor molecule and a strand transfer reaction is catalyzed by said integrase, wherein
   (a) said target DNA is fragmented to generate a plurality of target DNA fragments, and
   (b) said at least one DNA adaptor molecule is joined to at least one end of each of the plurality of said target DNA fragments,
      to generate a plurality of tagged DNA fragments of said target DNA.

The present invention also provides for the use of an integrase for generating a library of tagged DNA fragments of a target DNA.

The inventors have surprisingly realized that an integrase enzyme can be used to generate tagged DNA fragments of a target DNA or to generate a library consisting of such tagged DNA fragments. In contrast to the transposase-based fragmentation with simultaneous adaptor ligation as disclosed in WO 2010/048605 the method according to the invention offers a solution with better coverage evenness due to the less selectivity of the integrases in comparison with the transposases. The enzymatic integrase reaction is less complex and the strand transfer or integration of the DNA adaptor molecule does not alter the nucleotide sequences, thus ensuring high precision in a subsequent sequencing reaction. The integrase recognition sites are shorter than the transposase recognition sites making the tagged DNA fragments or the resulting library thereof more flexible.

The method according to the invention will allow the generation of a plurality of tagged DNA fragments or a library in only one step and will reduce the working time from a day to one to two hours. The obtained tagged DNA fragments can be used in different NGS platforms.

As used herein, “target DNA” refers to any double-stranded DNA (dsDNA) of interest that is subjected to the reaction mixture for generating tagged fragments thereof. “Target DNA” can be derived from any in vivo or in vitro source, including from one or multiple cells, tissues, organs, or organisms, whether living or dead, whether prokaryotic or eukaryotic, or from any biological or environmental source. Typically but not exclusively, “target DNA” refers to such dsDNA the nucleotide sequence is to be elucidated by sequencing, e.g. next generation sequencing (NGS).

As used herein, a “DNA fragment” means a portion or piece or segment of a target DNA that is cleaved from or released or broken from a longer DNA molecule such that it is no longer attached to the parent molecule.

As used herein, an “integrase” refers to a protein having the enzymatic activity of retroviral integrase produced by a retrovirus, such as HIV. It enables integrating a DNA adaptor molecule preferably via its integrase recognition site into the target DNA by 3′processing of the DNA adaptor molecule or integrase recognition site, respectively, and the transfer of the DNA adaptor molecule to the target DNA, thus, generating tagged DNA fragments of the target DNA.

As used herein, the “DNA adaptor molecule” refers to a dsDNA molecule to be joined to one or both extremities of the fragments of a target DNA in order to provide for the tagging. Typically, a “DNA adaptor molecule” has a length of between approximately 5 to 100 bp. Therefore, the “DNA adaptor molecule” cannot be equated with the dsDNA transposon-like molecules as e.g. used in the WO 2010/048605 A1.

As used herein, “integrase-recognition site” refers to a section or sequence of dsDNA or the DNA adaptor molecule, respectively, which is specifically and selectively recognized and bound by the integrase, thus allowing the integration and/or transfer of the DNA adaptor molecule to the target DNA. The “integrase-recognition site [[side]]” includes or can be embodied by nucleotide sequences called long terminal repeats (LTR).

As used herein, “tagged” refers to the process of joining the DNA adaptor molecule to the target-DNA molecule. DNA that undergoes tagging or that contains tag is referred to as “tagged”, e.g. “tagged DNA”.

The conditions wherein a “processing of said at least one DNA adaptor molecule and a strand transfer reaction are catalyzed” are well-known to the skilled person. Such conditions provide an environment for the integrase allowing the latter to exert its enzymatic activity. Such conditions further ensure that the integrase, the target DNA and the DNA adapter molecule will be able to interact to allow the integrase reaction.

The generation of tagged DNA fragments or plurality of DNA fragments also includes the concept of the generation of a library of tagged fragments.

The method according to the invention is far from being obvious.

So far, the principle of integration of viral nucleic acid into host DNA is only used to develop assays that can be employed in testing the activity of integrases and its inhibitors. In this context reference is made to the following publications dealing with HIV integrase type 1: lnayoshi et al. (2010), Transcription factor YY1 interacts with retroviral integrases and facilitates integration of moloney murine leukemia virus cDNA into the host chromosomes, J. Virol. 84(16), p. 8250-8261; Goodarzi et al. (1995), Concerted integration of retrovirus-like DNA by human immunodeficiency virus type 1 integrase, J. Virol. 69(10), p. 6090-6097; Ellison et al. (1994), A stable complex between integrase and viral DNA ends mediates human immunodeficiency virus integration in vitro, Proc. Natl. Acad. Sci. USA 91(15), p. 7316-7320; Yoshinaga et al. (1995), Different roles of bases within the integration signal sequence of human immunodeficiency virus type 1 in vitro, J. Virol. 69(5), p.3233-3236; Quashie et al. (2012), Novel therapeutic strategies targeting HIV integrase, BMC Med. 10, p. 34; Pruss et al. (1994), Human immunodeficiency virus integrase directs integration to sites of severe DNA distortion within the nucleosome core, Proc. Natl. Acad. Sci. USA 91(13), p. 5913-5917; Tsuruyama et al. (2010), In vitro HIV-1 selective integration into the target sequence and decoy-effect of the modified sequence, PLoS One. 5(11), e13841; Hansen et al. (1999), Integration complexes derived from HIV vectors for rapid assays in vitro, Nat. Biotechnol. 17(6), p. 578-582; Delelis et al. (2008), Integrase and integration: biochemical activities of HIV-1 integrase, Retrovirology 5, p. 114.

The following publications refer to AMV integrase: Narezkina et al. (2004), Genome-wide analyses of avian sarcoma virus integration sites, J. Virol. 78(21), p. 11656-11663; Yao et al. (2003), Avian retrovirus integrase-enhanced transgene integration into mammalian cell DNA in vivo, Biotechniques 35(5), p. 1072-1078.

The following publication is focused on the integrase of the Visna Virus: Katzman et al. (1994), In vitro activities of purified visna virus integrase, J. Virol. 68(6), p. 3558-3569.

The following publication refers to the integrase of M-MuLV: Dildine et al. (1998), A chimeric Ty3/moloney murine leukemia virus integrase protein is active in vivo, J. Virol. 72(5), p. 4297-4307.

The so-called ZAM integrase is the subject of the following publication: Faye et al. (2008), Functional characteristics of a highly specific integrase encoded by an LTR-retrotransposon, PLoS One. 3(9), e3185.

HIV, AMV, MuLV integrases are the subject of the following publication: Dolan et al. (2009), Defining the DNA substrate binding sites on HIV-1 integrase, J. Mol. Biol. 385(2), p. 568-579.

However, the prior art is silent on the use of an integrase for generating tagged DNA fragments of a target DNA or a library consisting thereof, respectively.

The object underlying the invention is herewith completely solved.

According to a further development of the method of the invention in step (ii) (b) said at least one DNA adaptor molecule is joined to both ends of each of the plurality of said target DNA fragments.

This measure has the advantage that the tagged DNA fragments of the target DNA will be provided in a form ready to be processed in a subsequent reaction, e.g. a sequencing reaction by NGS.

According to a preferred embodiment of the method of the invention said at least one DNA adaptor molecule further comprises a site for annealing an oligonucleotide, preferably a PCR and/or sequencing primer (“primer annealing site”, PAS).

This measure has the advantage, that the tagged DNA fragments are already provided in a “ready-for-amplifying” or “ready-for-sequencing” condition.

The site for annealing an oligonucleotide can be configured to anneal an oligonucleotide primer for extension by a DNA polymerase, for example within the context of a next generation sequencing reaction (NGS), or to anneal an oligonucleotide for capture or for a ligation reaction. The DNA adaptor molecule may comprise the integrase recognition site or LTR, respectively, spaced apart from the annealing site, e.g. the integrase recognition site at its first end and the annealing site at its second end.

According to another embodiment the method of the invention is further comprising after step (ii) the following step: (ii)' subjecting said plurality of tagged DNA fragments of said target DNA to a PCR to add to said at least one DNA adaptor molecule a site for annealing an oligonucleotide, preferably said site for annealing an oligonucleotide is configured for annealing a PCR and/or sequencing primer.

By this alternative approach a DNA adaptor molecule can be used which only comprises the integrase recognition site (IRS). After having obtained the tagged DNA fragments the latter are incubated with at least one PCR primer pair configured to add in a PCR reaction to said at least one DNA adaptor molecule a site for annealing an oligonucleotide, such as a PCR and/or sequencing primer. The first PCR primer of said PCR primer pair may also comprise an IRS that can hybridize to the IRS of said DNA adaptor molecule. The first PCR primer may further comprise a site for annealing an oligonucleotide such as a PCR and/or sequencing primer. The second PCR primer of said PCR primer pair may then be configured to hybridize to the first PCR primer, preferably to the site for annealing an oligonucleotide. The first PCR primer of said PCR primer pair might therefore be longer than the second PCR primer. Subjecting said reaction mixture comprising the tagged DNA fragments and the at least one PCR primer pair (long and short PCR primer) to a PCR under conditions appropriate to amplify the tagged DNA fragments will result in an enrichment of the tagged DNA fragments. In parallel the DNA adaptor molecules of the tagged DNA fragments will then be completed by adding a site for annealing an oligonucleotide in the PCR.

According to a preferred embodiment of the method of the invention said integrase is selected from the group consisting of: retroviral integrases, including HIV integrases, and integrases derived from retroviral integrases.

This measure has the advantage that such an integrase is provided which has been proven to provide optimum results. Other suitable integrases are AMV integrase, Visna Virus integrase, MuLV integrase, ZAM integrase.

As used herein, “integrases derived from retroviral integrases” refers to a group of enzymes having the 3′ processing and strand transfer activity of a retroviral integrase. According to the invention integrases derived from retroviral integrases also encompass such integrases which comprise the so-called DDE motif that is essential for the catalysis of integration. Such derived integrases might be devoid of non-functional domains. An example of such a derived integrase is a HIV-1-derived integrase which has been used by the inventors. It comprises the “core” of the HIV-1 integrase that consists of amino acid numbers 50 to 212, but lacks the initial N terminal and the final C terminal amino acids.

According to a preferred further development of the method of the invention said at least one DNA adaptor molecule is consisting of two nucleic acid molecules comprising complementary nucleotide sequences and being specifically hybridized to each other, selected from the following group:

Adaptor 1 (SEQ ID no. 1+SEQ ID no. 2),

Adaptor 2 (SEQ ID no. 3+SEQ ID no. 2),

Adaptor 3 (SEQ ID no. 6+SEQ ID no. 2),

Adaptor 4 (SEQ ID no. 7+SEQ ID no. 2),

Adaptor 5 (SEQ ID no. 8+SEQ ID no. 4),

Adaptor 6 (SEQ ID no. 9+SEQ ID no. 4),

Adaptor 7 (SEQ ID no. 8+SEQ ID no. 5),

Adaptor 8 (SEQ ID no. 9+SEQ ID no. 5),

Adaptor 9 (SEQ ID no. 14+SEQ ID no. 15),

Adaptor 10 (SEQ ID no. 10+SEQ ID no. 11),

Adaptor 11 (SEQ ID no. 12+SEQ ID no. 13).

This measure has the advantage that such DNA adaptor molecules are provided which are particularly suited for the method according to the invention.

It is agreed that the first sequence recited in brackets refers to the first strand of the dsDNA adaptor sequence and the second sequence recited in brackets refers to the second strand of the dsDNA adaptor molecule.

According to a further development of the method of the invention it further comprises
(iii) Purifying said plurality of tagged DNA fragments of that target DNA.

Such measure has the advantage that the integrase, non-fragmented target DNA, non-tagged fragments of target DNA and adaptor DNA etc. are removed, for example by using QlAquick columns, thereby providing a purified library of tagged DNA fragments for the further use.

It is preferred if the method according to the invention is performed within one reaction vessel.

This measure embodies the principle of a “one-step” method. Even though the method according to the invention is subdivided in (i), (ii), and (iii). This sub-division only intends to illustrate the chronological sequence of the method events. However, the user of the method is only required to create the reaction mixture under the prescribed conditions, thereby automatically producing the plurality or library of tagged DNA fragments of the target DNA in one step.

Another subject matter of the present invention relates to a kit for generating tagged DNA fragments of a target DNA, comprising:
(i) an integrase, and
(ii) at least one DNA adaptor molecule that comprises an integrase recognition site.

As for the method according to the invention, said at least one DNA adaptor molecule further comprises a site for annealing an oligonucleotide, preferably for annealing a PCR and/or sequencing primer.

The integrase contained in the kit of the invention is selected from the group consisting of: retroviral integrases, including HIV integrases, integrases derived from retroviral integrases. Other suitable integrases are AMV integrase, Visna Virus integrase, MuLV integrase, ZAM integrase.

According to a further development of the kit of the invention said at least one DNA adaptor molecule is consisting of two nucleic acid molecules comprising complementary nucleotide sequences and being specifically hybridized to each other, selected from the following group:

Adaptor 1 (SEQ ID no. 1+SEQ ID no. 2),

Adaptor 2 (SEQ ID no. 3+SEQ ID no. 2),

Adaptor 3 (SEQ ID no. 6+SEQ ID no. 2),

Adaptor 4 (SEQ ID no. 7+SEQ ID no. 2),

Adaptor 5 (SEQ ID no. 8+SEQ ID no. 4),

Adaptor 6 (SEQ ID no. 9+SEQ ID no. 4),

Adaptor 7 (SEQ ID no. 8+SEQ ID no. 5),

Adaptor 8 (SEQ ID no. 9+SEQ ID no. 5),

Adaptor 9 (SEQ ID no. 14+SEQ ID no. 15),

Adaptor 10 (SEQ ID no. 10+SEQ ID no. 11),

Adaptor 11 (SEQ ID no. 12+SEQ ID no. 13).

A kit is a combination of individual elements useful for carrying out the method of the invention, wherein the elements are optimized for use together in the method. The kit also contains a manual for performing the method according to the invention. Such a kit unifies all essential elements required to work the method according to the invention, thus minimizing the risk of errors. Therefore such kit also allows semi-skilled laboratory staff to perform the method according to the invention.

The features, characteristics, and advantages of the method according to the invention apply mutatis mutandis to the kit and use according to the invention, respectively.

The kit according to the invention may comprise more than one, e.g. two, three or four or more different integrases as well as more than one DNA adapter molecule, e.g. two, three, four, etc. different DNA adapter molecules. The kit can also contain one or different buffer compositions, to create an optimum environment for the integrase and the integration reaction, a reference target DNA, etc.

Another subject matter of the present invention relates to a nucleic acid molecule comprising a nucleotide sequence selected from the group consisting of SEQ ID no. 1 to 15.

The nucleic acid molecule according to the invention is specifically adapted to be used in the method of the invention. In particular, the nucleic acid molecules can be hybridized to each other in order to form DNA adaptor molecules suitable for a direct use in the method according to the invention. The hybridization schedule is as follows:

SEQ ID no. 1+SEQ ID no. 2: adaptor 1

SEQ ID no. 3+SEQ ID no. 2: adaptor 2

SEQ ID no. 6+SEQ ID no. 2: adaptor 3

SEQ ID no. 7+SEQ ID no. 2: adaptor 4

SEQ ID no. 8+SEQ ID no. 4: adaptor 5

SEQ ID no. 9+SEQ ID no. 4: adaptor 6

SEQ ID no. 8+SEQ ID no. 5: adaptor 7

SEQ ID no. 9+SEQ ID no. 5: adaptor 8

SEQ ID no. 14+SEQ ID no. 15: adaptor 9

SEQ ID no. 10+SEQ ID no. 11: adaptor 10

SEQ ID no. 12+SEQ ID no. 13: adaptor 11

Unless defined otherwise, all technical and scientific terms used herein generally have the same meaning as commonly understood by one of ordinary skill in the art to which this invention belongs.

It goes without saying that the above-mentioned features and the features which are still to be explained below can be used not only in the respective specified combinations, but also in other combinations or on their own, without departing from the scope of the present invention.

Further feature, characteristics and advantages follow from the description of preferred embodiments and the attached figures.

### IN THE FIGURES:

FIG. 1 shows a diagram illustrating the differences in the sequence of events in HIV-1 integration involving integrases (left) and Tn5 transposition involving transposases (right).

FIG. 2 shows a diagram illustrating an embodiment of the method according to the invention (A) and details on a tagged plasmid DNA fragment generated by said method (B).

FIG. 3 shows photographs of agarose gels demonstrating the successful generation of tagged plasmid DNA fragments by the method of the invention.

FIG. 4 shows an electropherogramm of fragmented and adaptor ligated genomic DNA using two different cycling conditions and two different reaction buffers.

FIG. 5 shows a diagram illustrating another embodiment of the method according to the invention.

FIG. 6 shows electropherogramms of fragmented and adaptor ligated genomic DNA using various fragmentation adaptors and PCR primer mixes.

FIG. 7 shows electropherogramms of fragmented and adaptor ligated genomic DNA using different incubation temperatures.

### EXAMPLES

A central aspect of the method according to the invention is the use of an integrase enzyme in contrast to the use of a transposase enzyme employed in the prior art fragmentation and simultaneous adaptor ligation, e.g. as disclosed in WO 2010/048605.

Integration of retrovitral DNA is an obligatory step of retrovirus replication because proviral DNA is the template for productive infection. The process of integration as catalyzed by the integrase can be divided into two sequential reactions. The first one, named 3′ processing, corresponds to a specific endonucleolytic reaction which prepares the viral DNA extremities to be competent for the subsequent covalent insertion, named strand transfer, into the host cell genome by a trans-esterification reaction. The integrase first binds to a short sequence at each and of the viral DNA known as integrase recognition sequence (IRS) or long terminal repeat (LTR), respectively, and catalyzes an endonucleotide cleavage known as 3′ processing, in which a denucleotide is eliminated from each and of the viral DNA. The resulting cleaved DNA is then used as substrate for integration or strand transfer leading to the covalent insertion of the viral DNA into the genome of the infected cell. This second reaction occurs simultaneously at both ends of the viral DNA molecule, with an offset of precisely five base pairs between the two opposite points of insertion.

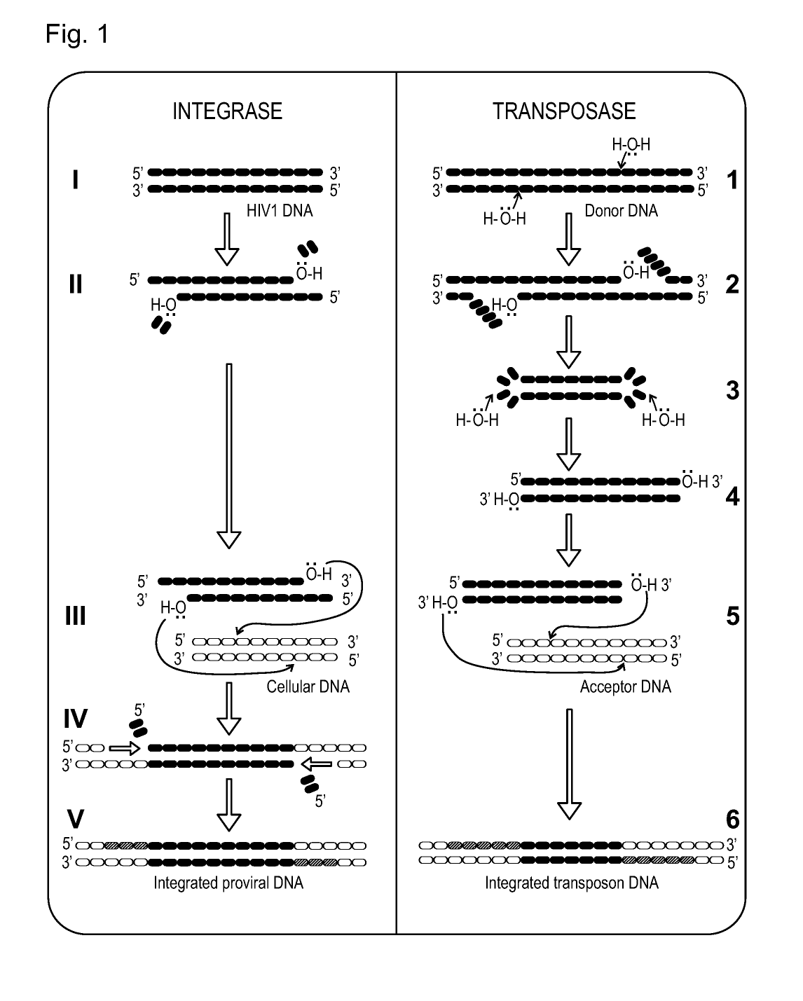

In FIG. 1 such events are illustrated in HIV-1 integration (left) in comparison with Tn5 transposition (right). HIV-1: I) donor DNA; II) integrase-catalyzed 3′ processing; III) integrase-catalyzed strand transfer; IV) product of strand transfer; V) DNA repaired strand transfer product. Tn5 transposon: 1) donor DNA; 2) 3′ processing; 3-4) 5′ processing, consisting of loop formation (3) and generation of blunt-ended DNA (4); 5) strand transfer; 6) repaired strand transfer product.

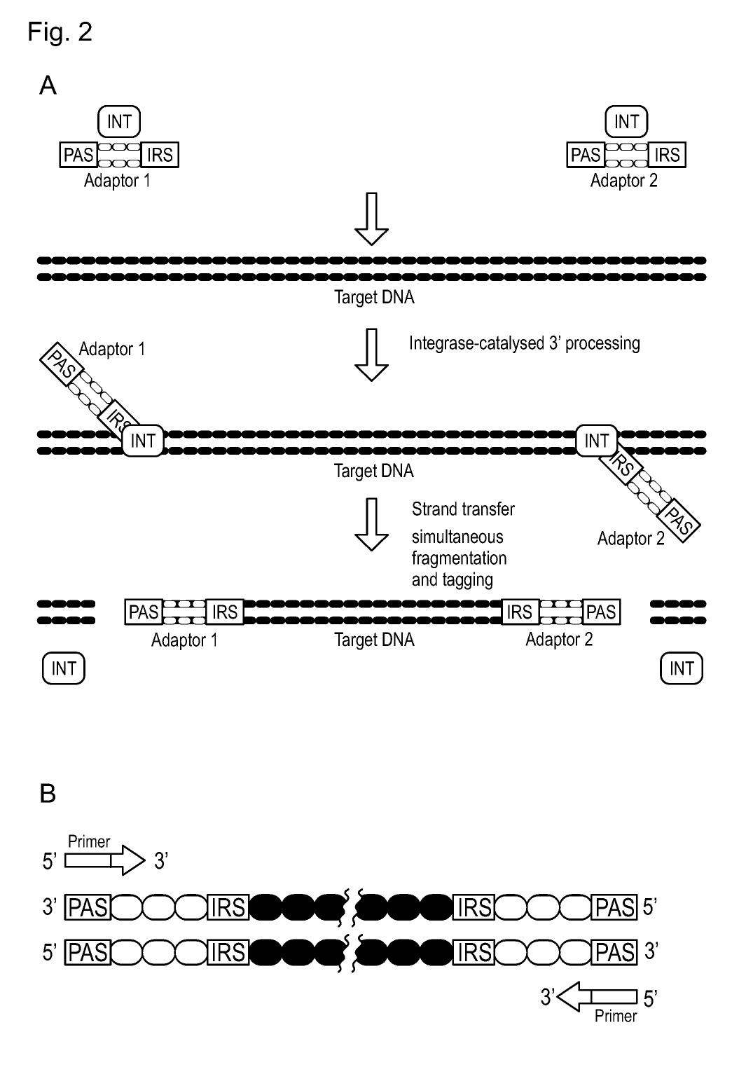

FIG. 2 shows a graphical illustration of the method according to the invention. Two DNA adaptor molecules (Adaptors 1 and 2) each of which comprising an integrase recognition site (IRS) and a primer annealing site (PAS), were incubated with an integrase enzyme (INT) and the target DNA to be fragmented and tagged; cf. FIG. 2A upper part.

The integrase (INT) binds to the IRS of the adaptor molecules Adaptor 1 and 2 and the target DNA and catalyzes the 3′ processing and strand transfer; cf. FIG. 2A, middle part.

In FIG. 2A, lower part, the result of the integrase reaction is shown, i.e. the fragmented and tagged target DNA having at its both ends joined adaptors, wherein the adaptors are joined via the respective IRS sections of the adaptors thus exposing the PASs at the extremities of the fragmented and tagged target DNA.

In FIG. 2B the fragmented and tagged target DNA is shown in further detail. The fragmented and tagged target DNA comprises at its extremities the PAS sections allowing the annealing of a PCR primer and the elongation of the latter in 3′ direction.

The integrase reaction is used in the method of the present invention to fragment genomic DNA and ligate DNA adaptor molecules to both ends. The DNA adaptor molecules then can be used for e.g. amplification of the generated tagged and fragmented target DNA and subsequently cluster generation and sequencing.

Two different HIV integrases were exemplarily used, namely a codon optimized, in-house expressed and purified HIV-1-derived integrase having 171 amino acids of the sequence as shown under SEQ ID no. 16. The HIV-1-derived integrase has a size of 18.97 kDa and comprises the core domain of HIV integrase represented by amino acids numbers 50 to 212. Such integrase is referred to as “QHIN 1”. The HIV-1-derived integrase catalyzes the disintegration reaction, however not the integration (3′ processing and transfer). ∈=27965; pl (theoretically): pH 7.82; mutation: F185K (solubility).

The second integrase is a commercially available wild-type HIV-1 integrase (BioProducts MD, LLC, Middletown, Md., United States of America). Such integrase is referred to as “BPHIN 1”.

Different adaptor molecules were designed to include recognition sides for the HIV-1 integrase and sequences that can be used for the amplification of the library and subsequently sequencing on Illumina NGS platforms. The following Table 1 includes the sequences that were used by the inventors to form the DNA adapter molecules:

TABLE 1
Sequences used for the
generation of DNA adaptor molecules

| | | SEQ |
|---|---|---|
| | | ID |
| Name | Sequence | no. |
| 21/21_IN_1 | 5′GTGTGGAAAATCTCTAGCAGT-3′ | 1 |
| 21/21_IN_2 | 5′-ACTGCTAGAGATTTTCCACAC-3′ | 2 |
| 19/21_IN_3 | 5′GTGTGGAAAATCTCTAGCA-3′ | 3 |
| rev_6(long)_ | 5′-ACTGCT(AGATCGGAAGTGC)-3′ | 4 |
| IN_10 | | |
| rev_6_IN_11 | 5′-ACTGCT-3′ | 5 |
| 21/21plus_IN_4 | 5′ AAT GAT ACG GCG ACC ACC GAG | 6 |
| | ATC TAC ACT CTT TCC CTA CAC GAC | |
| | GCT CTT CCG ATC TGTGTGGAAAATCTC | |
| | TAGCAGT-3′ | |
| 21/21plus_IN_5 | 5′-CAA GCA GAA GAC GGC ATA CGA | 7 |
| | GAT CGT GAT GTG ACT GGA GTT CAG | |
| | ACG TGT GCT CTT CCG ATC TGTGTGG | |
| | AAAATCTCTAGCAGT-3′ | |
| 6/6plus_IN_6 | 5′ AAT GAT ACG GCG ACC ACC GAG | 8 |
| | ATC TAC ACT CTT TCC CTA CAC GAC | |
| | GCT CTT CCG ATC TAGCAGT-3′ | |
| 6/6plus_IN_7 | 5′-CAA GCA GAA GAC GGC ATA CGA | 9 |
| | GAT CGT GAT GTG ACT GGA GTT CAG | |
| | ACG TGT GCT CTT CCG ATC TAGCAG | |
| | T-3′ | |
| yoshi.U5LTR | 5′-TGT GTG CCC GTC TGT TGT GTG | 10 |
| | ACT CTG GTA ACT AGA GAT CCT CAG | |
| | ACC TTT TTG GTA GTG TGG AAA ATC | |
| | TCT AGC A-3′ | |
| yoshi.U5LTR- | 5′-ACT GCT AGA GAT TTT CCA CAC | 11 |
| revB | TAC CAA AAA GGT CTG AGG ATC TCT | |
| | AGT TAC CAG AGT CAC ACA ACA GAC | |
| | GGG CAC ACA-3′ | |
| yoshi.U3LTR | 5′-ACT GGA AGG GTT AAT TTA CTC | 12 |
| | CAA GCA AAG GCA AGA TAT CC TTG | |
| | ATT TGT GGG TCT ATA ACA CAC AAG | |
| | GCT ACT TCC CA-3′ | |
| yoshi.U3LTR- | 5′-TGG GAA GTA GCC TTG TGT GTT | 13 |
| rev | ATA GAC CCA CAA ATC AAG GAT ATC | |
| | TTG CCT TTG CTT GGA GTA AAT TAA | |
| | CCC TTC CAG T-3′ | |
| RB67_IN_8 | 5′-CGA TAG GAT CCG AGT GAA TTA | 14 |
| | GCC CTT CCA-3′ | |
| RB50_IN_9 | 5′-AC TGG AAG GGC TAA TTC ACT | 15 |
| | CGG ATC CTA TCG-3′ | |

Adaptor molecules were formed by mixing the before-listed oligonucleotides in different ratios to each other. An initial denaturation step of two minutes at 98° C. to eliminate putative secondary structures of the oligonucleotides was followed by a slow cooling down of the probes to allow annealing of the complementary oligonucleotides. The following Table 2 shows the different adaptors formulations.

TABLE 2
DNA adaptor molecules

| Dilute in RNAse | | | |
|---|---|---|---|
| free Water | Mix | And | Ratio |
| IN adaptor 1 | 21/21_IN_1 (SEQ ID no. 1) | 21/21_IN_2 (SEQ ID no. 2) | 1:2 |
| IN adaptor 2 | 19/21 IN_3 (SEQ ID no. 3) | 21/21_IN_2 (SEQ ID no. 2) | 1:2 |
| IN adaptor 3 | 21/21plus_IN_4 (SEQ ID no. 6) | 21/21_IN_2 (SEQ ID no. 2) | 1:2 |
| IN adaptor 4 | 21/21plus_IN_5 (SEQ ID no. 7) | 21/21_IN_2 (SEQ ID no. 2) | 1:2 |
| IN adaptor 3 | 21/21plus_IN_4 (SEQ ID no. 6) | 21/21_IN_2 (SEQ ID no. 2) | 1:4 |
| IN adaptor 4 | 21/21plus_IN_5 (SEQ ID no. 7) | 21/21_IN_2 (SEQ ID no. 2) | 1:4 |
| IN adaptor 5 | 6/6plus_IN_6 (SEQ ID no. 8) | rev_6(long)_IN_10 (SEQ ID no. 4) | 1:2 |
| IN adaptor 6 | 6/6plus_IN_7 (SEQ ID no. 9) | rev_6(long)_IN_10 (SEQ ID no. 4) | 1:2 |
| IN adaptor 5 | 6/6plus_IN_6 (SEQ ID no. 8) | rev_6(long)_IN_10 (SEQ ID no. 4) | 1:4 |
| IN adaptor 6 | 6/6plus_IN_7 (SEQ ID no. 9) | rev_6(long)_IN_10 (SEQ ID no. 4) | 1:4 |
| IN adaptor 7 | 6/6plus_IN_6 (SEQ ID no. 8) | rev_6_IN_11 (SEQ ID no. 5) | 1:2 |
| IN adaptor 8 | 6/6plus_IN_7 (SEQ ID no. 9) | rev_6_IN_11 (SEQ ID no. 5) | 1:2 |
| IN adaptor 7 | 6/6plus_IN_6 (SEQ ID no. 8) | rev_6_IN_11 (SEQ ID no. 5) | 1:4 |
| IN adaptor 8 | 6/6plus_IN_7 (SEQ ID no. 9) | rev_6_IN_11 (SEQ ID no. 5) | 1:4 |
| IN adaptor 9 | RB67_IN_8 (SEQ ID no. 14) | RB50_IN_9 (SEQ ID no. 15) | 1:2 |
| IN adaptor 10 | yoshi.U5LTR (SEQ ID no. 10) | yoshi.U5LTR-revB (SEQ ID no. 11) | 1:2 |
| IN adaptor 11 | yoshi. U3LTR (SEQ ID no. 12) | yoshi. U3LTR-rev (SEQ ID no. 13) | 1:2 |

In a first feasibility assay the IN adaptors 1 to 11 were used in combination with the codon-optimized, in-house expressed HIV-1-derived integrase (QHIN 1) to simultaneously fragment and adaptor ligate a bacterial plasmid DNA (pGL2).

### The Experimental Schedule is as Follows:

| | Reagent | conc. in RXN | μL |
|---|---|---|---|
| mix | QHIN 1 | 800 nM | 2 |
| | Integration Adaptor 10 μM | 50 nM | 0.25 |
| | Buffer 2x* | 1x | 25 |
| | Water | | 21.75 |

*Buffer 2x: 10 mM MnCl₂, 40 mM HEPES (pH 7.5), 2 mM dithiothreitol, 0.1% Nonidet P40, 1 mM CHAPS, 40 mM NaCl.

Incubation for 10 min at 37° C. to form the integration complexes.

| | Reagent | conc. in RXN | μl |
|---|---|---|---|
| add | Target DNA (Plasmid; pGL2) 274 ng/μL | 50 nM | 1 |
| | Total | | 50 |

Incubation for 1 h at 37° C. for the fragmentation and simultaneous adaptor ligation of the plasmid target DNA.

After the incubation the fragmented and adaptor ligated DNA was purified using QlAquick columns and reaction clean-up protocol. The agarose gel analysis showed no fragments since the concentrations of plasmid and fragments are too low to be visualized on an agarose gel. Fragmented and adaptor ligated DNA was then amplified using specific primers for the adaptors. For IN adaptor 1 and 2 no PCR primers have been available. For IN adaptors 3 to 8 the IIlumina P1 and P2 primers were used, for IN adaptor 9 the RB primer and for IN adaptors 10 and 11 the U5LTR and U3LTD primers were used.

In the following table the sequences of the used PCR primers are listed.

TABLE 3
Used PCR primers

| | | SEQ |
|---|---|---|
| PCR Primers: | Sequence | ID no. |
| Primer P1 | AAT GAT ACG GCG ACC ACC GA | 17 |
| Primer P2 | CAA GCA GAA GAC GGC ATA CGA | 18 |
| U5LTR For | GTGTGCCCGTCTGTTGTGT | 19 |
| U5LTR Rev | CCACACTACCAAAAAGGTCTGA | 20 |
| U3LTR For | ACTCCAAGCAAAGGCAAGAT | 21 |
| U3LTR Rev | TGGGAAGTAGCCTTGTGTGTT | 22 |
| RB Primer | AG GAT CCG AGT GAA TTA GCC CT | 23 |

PCR set-up protocol and cycling conditions are listed below.

| MMX | Conc. | Conc. in RXN | μL |
|---|---|---|---|
| HotStarTq MMX 2x | | 1x | 25 |
| Primer for | 10 μM | 0.3 μM | 1.5 |
| Primer rev | 10 μM | 0.3 μM | 1.5 |
| or Primer Mix | 10 μM | 0.3 μM | 3 |
| Template | | | 5 |
| Rnase Free Water | | | 17 |
| Volume Total | | | 50 |

| Cycling | | | |
|---|---|---|---|
| 95° C. | 15 | min | |
| 94° C. | 30 | sec | 35x |
| 60° C. | 30 | sec | |
| 72° C. | 1 | min | |
| 72° C. | 10 | min | |

| | |
|---|---|
| 4° C. | hold |

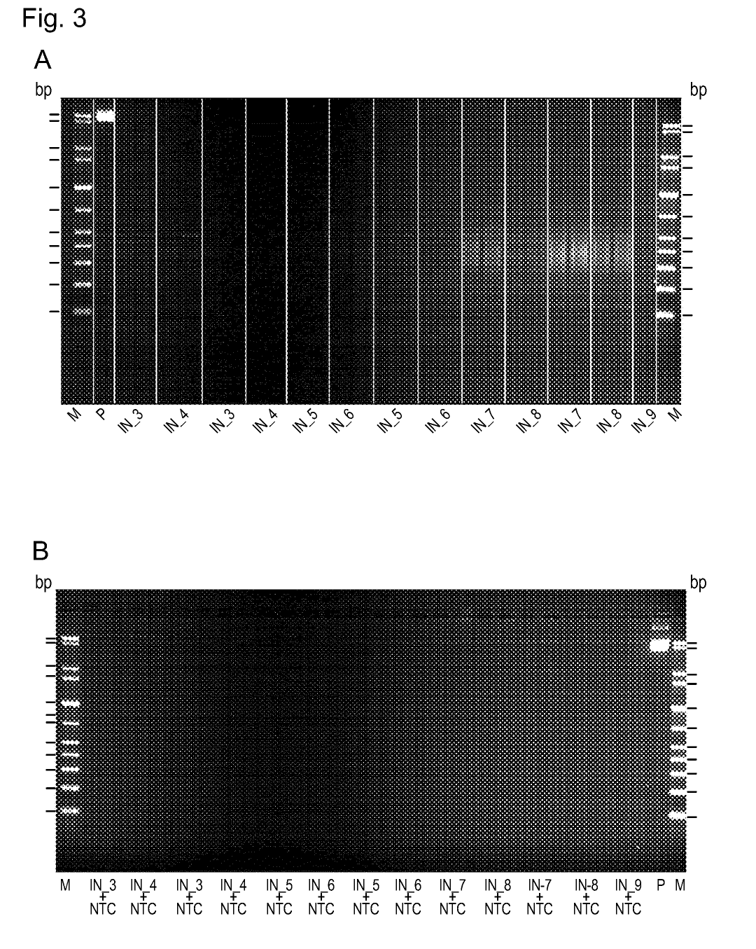

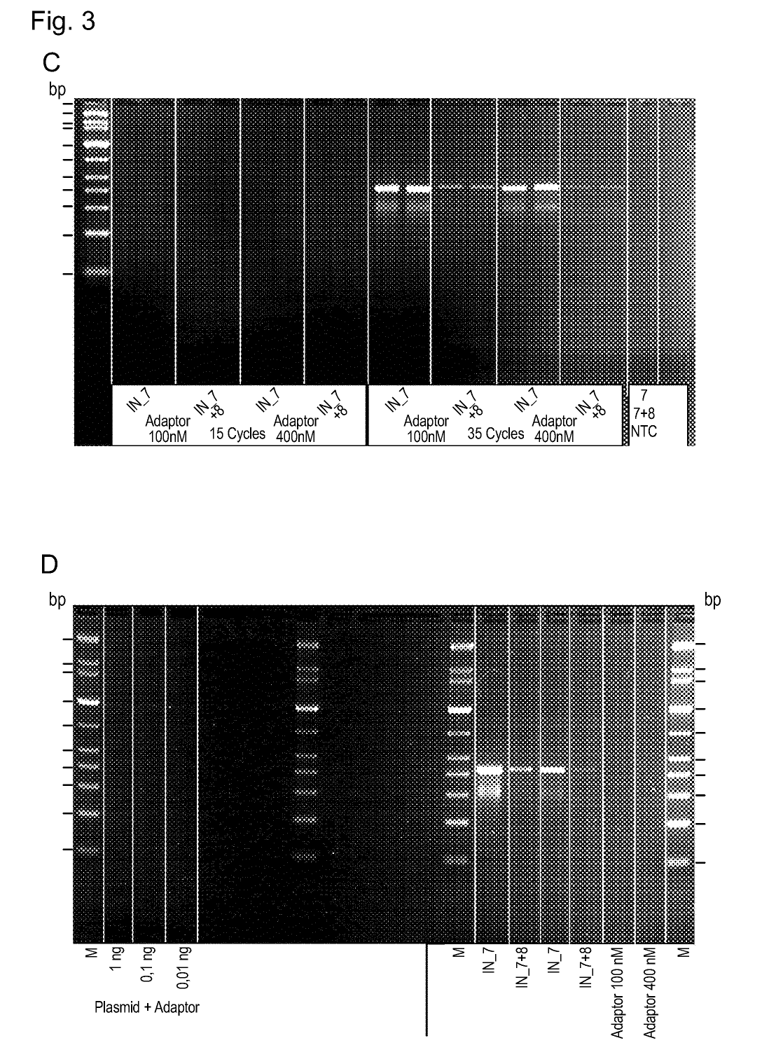

The amplicons were analyzed in a 2% agarose gel and show a fragmentation of the plasmid DNA with sizes between 250 and 1000 bp (FIG. 3A).

In order to see if the fragmentation is an effect caused by the remaining adaptors in the PCR a second PCR was performed with the same fragmented and ligated samples and PCR only with the adaptors as “no template control” (NTC). No amplicons were obtained using only the adaptors (NTC). FIG. 3B shows the agarose gel by means of which the amplified fragmented and ligated DNA is analyzed side by side with the corresponding adaptors amplification (NTC).

In a second experiment a second HIV-1 integrase, (wild-type HIV integrase; Bio Products MD, LLC, Middletown, MD, USA) (BPHIN 1) was used to fragment and ligate plasmid DNA (pGL2) using the best performing adaptors. IN adaptor 7 and IN adaptor 7 in pair with 8 were used in this assay.

### Assay

| | Reagent | conc. in RXN | μL |
|---|---|---|---|
| mix | BPHIN 1 | 3.2 nM | 5 |
| | IN adaptor 10 μM | 400 nM | 2 |
| | Buffer 2x* | 1x | 25 |
| | Water | | 13.74 |

*Buffer 2x: 10 mM MnCl₂, 40 mM HEPES (pH 7.5), 2 mM dithiothreitol, 0.1% Nonidet P40, 1 mM CHAPS, 40 mM NaCl.

Incubate 10 min at 37° C.

| | Reagent | conc. in RXN | μl |
|---|---|---|---|
| add | Target (Plasmid); pGL2 274 ng/μL | 50 nM | 4.26 |
| | total | | 50 |

Incubate for 1 h at 37° C.

The fragmented and adaptor ligated target DNA was amplified using the Illumina primers P1 and P2 and analyzed on an agarose gel. The result is shown in FIG. 3C. Again, a fragmentation of the plasmid was obtained with fragment sizes between 150 and 500 bp.

In order to test if these results are an artifact from non-specific plasmid amplification, the plasmid was amplified in parallel with the fragmented and adaptor ligated plasmid DNA using the P1 and P2 primers and analyzed in agarose electrophoresis. The result is shown in FIG. 3D. As can be seen, in the gel image no amplification was obtained with the plasmid and the adaptors.

After testing the invention by using plasmids as target DNA the next step was to test whether by the inventive method it was able to generate libraries using genomic DNA as target DNA. In the following experiments E. coli DNA was used as target DNA for the generation of fragmented DNA with adaptor on the fragment ends that can be used for amplification of these fragments and subsequent sequencing on NGS platforms.

For the following setup the best performing adaptors IN_adaptor_7 and IN_adaptor_8 were used. QHIN_1 stored in two different buffers (D and VV) was tested in parallel. 10Ong genomic DNA from E. coli was used as target DNA for fragmentation and adapter ligation.

### Experimental Setup:

QHIN_1 storage buffers

D: Dar-Buffer
25 mM Tris-HCl pH7,4
1 M NaCl
7,5 mM CHAPS
1 mM DTT
50% Glycerol

W: Wang50-Buffer
20 mM HEPES pH7,35
1 M NaCl
1 mM DTT
50% Glycerol
   Two mastermixes (MMX) were prepared one using 0.2 pM adaptor.

| Reagent | conc. in RXN | μL |
|---|---|---|
| QHIN 1 | 0.16 μM | 5 |
| IN adaptor 6 | 0.2 μM | 1 |
| IN adaptor 7 | 0.2 μM | 1 |
| Buffer 2x | 1x | 25.00 |
| Water | | 13.00 |
| incubate 10 min at 37° C. | Total | 45.00 |

| | | | |
|---|---|---|---|
| add | Target (gDNA) 100/20 ng/μL | 100 ng | 5.00 |
| | Total | | 50.00 |

After incubation the samples were purified with QiaQuick columns and PCR-amplified with Primers P1 and P2 using two different cycling conditions in order to investigate if completion of gaps in the strands resulted by the integration is needed before the conventional cycling.

### The PCR Setup is Described in the Following Table:

| Stock μM | final conc. μM | volume | |
|---|---|---|---|
| 2xMMX | 1x | 25 | μL |
| 10 μM Forward Primer | 0.3 μM | 1.5 | μL |
| 10 μM Reverse Primer | 0.3 μM | 1.5 | μL |
| 5x Q-Solution | 0.5x | 5 | μL |
| Template DNA | | 12 | μL |
| Water | | 5 | μL |
| Total | | 50 | μL |

### Cycling Conditions:

| 1. cycling: | 98° C. | 2 | min | |
|---|---|---|---|---|
| | 98° C. | 20 | sec | 35x |
| | 60° C. | 30 | sec | |
| | 72° C. | 30 | sec | |
| | 72° C. | 1 | min | |
| | 4° C. | hold | | |
| 2. cycling: | 98° C. | 2 | min | |
| | 72° C. | 2 | min | |
| | 98° C. | 20 | sec | 35x |
| | 60° C. | 30 | sec | |
| | 72° C. | 30 | sec | |
| | 72° C. | 1 | min | |

| | |
|---|---|
| 4° C. | hold |

After the PCR the remaining adaptors and primers were removed using Agencourt AMPure XP Beads and probes were analyzed via capillary electrophoresis using and Agilent DNA chip.

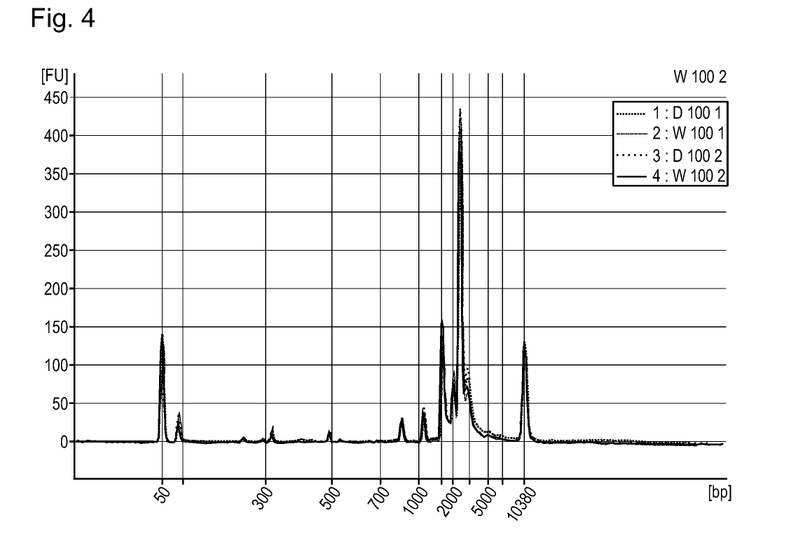

### FIG. 4 Represents the Electropherogram of all Samples:

1: D 100 1; Dar-Buffer/100 ng gDNA/1.cycling
2: W 100 1; Wang50-Buffer/100 ng gDNA/1.cycling
3: D 100 2; Dar-Buffer/100 ng gDNA/2.cycling
4: W 100 2; Wang50-Buffer/100 ng gDNA/2.cycling.

Here fragments of amplified DNA can be seen with a main size distribution between 1000-5000 bp. That means fragmentation and adaptor ligations occurred and the generated fragments could be amplified using Primers P1 and P2.

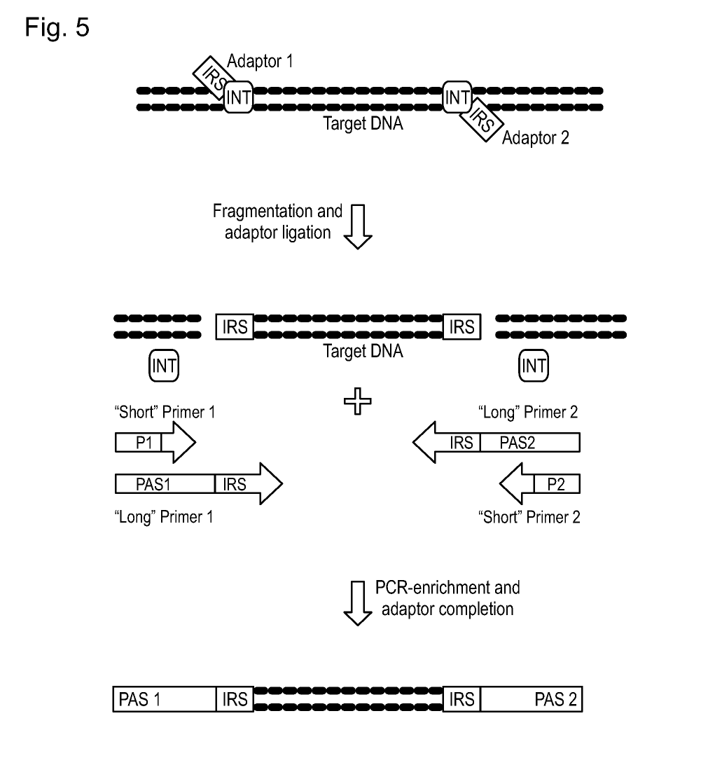

Further experiments were performed to optimize the size distributions of the fragments without giving different results (data not shown). That's why the inventors have tried to perform fragmentation using short adaptors only comprising the integrase recognition site (IRS) and then complete the adaptor sequence over PCR by adding the primer annealing site (PAS). The principle of this embodiment is illustrated in FIG. 5. After the target DNA has been simultaneously fragmented and adaptor ligated (upper part) the ligated fragments are then subjected to a PCR (lower part). In the PCR two PCR primer pairs are used. The first PCR primer pair is consisting two primers each of which comprising an integrase recognition site (IRS) capable to hybridize to the IRS of the adaptor ligated DNA fragments, and each comprising a primer annealing site (PAS1 or PAS2). The second PCR primer pair is consisting of two primers (P1 and P2) each of which can hybridize to the primer annealing sites PAS1 or PAS2, respectively. In the subsequent PCR the adaptor ligated DNA fragments are amplified and the adaptors are completed by addition of the primer annealing sites PAS1 and PAS2.

Therefore, for further fragmentation experiments the fragmentation adaptors comprising IRS but no PAS (IN_adaptor 1; IN_adaptor_2), the PCR primer mix-1 (21/21plus_IN_4 (SEQ ID no. 6); 21/21plus_IN_5 (SEQ ID no. 7); Primer P1 (SEQ ID no. 17); Primer P2 (SEQ ID no. 18), or PCR primer mix-2 (6/6plus_IN_6 (SEQ ID no. 8); 6/6plus_IN_7 (SEQ ID no. 9); Primer P1 (SEQ ID no. 17); Primer P2 (SEQ ID no. 18) were used. The “long” PCR primers 21/21plus_IN_4 and 21/21plus_IN_5 or 6/6plus_IN_6 and 6/6plus_IN_7 comprise the IRSs and PAS, respectively. The “short” PCR primers P1 and P2 can hybridize to the respective PAS.

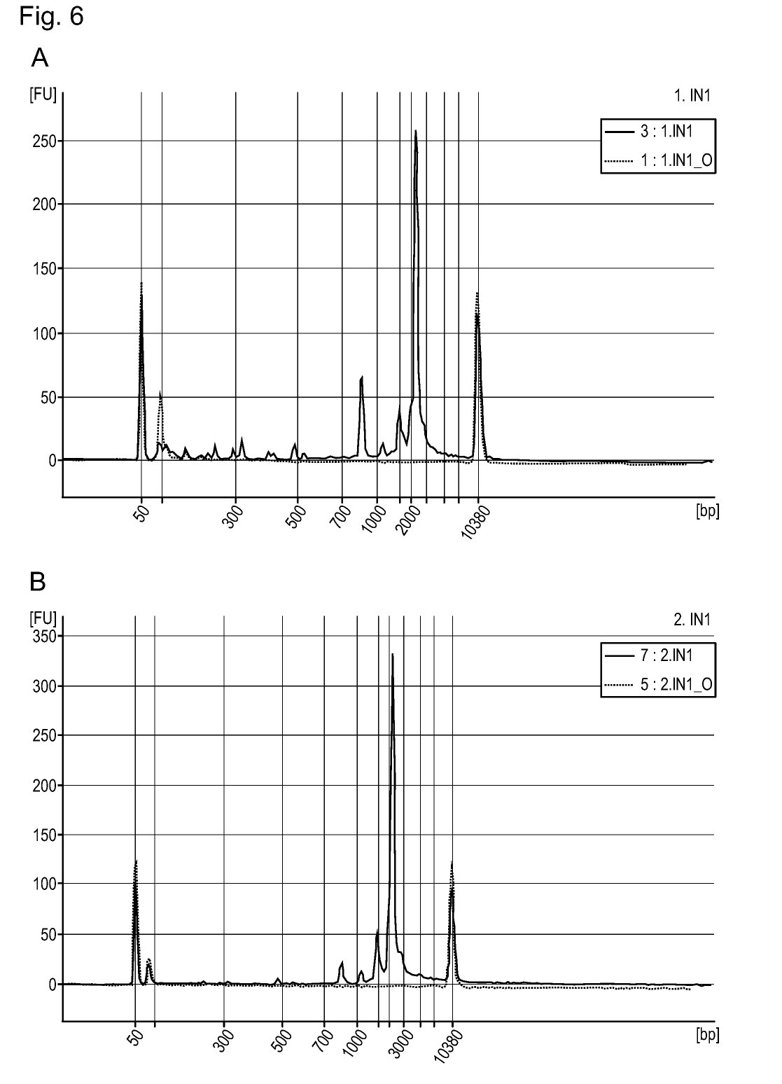

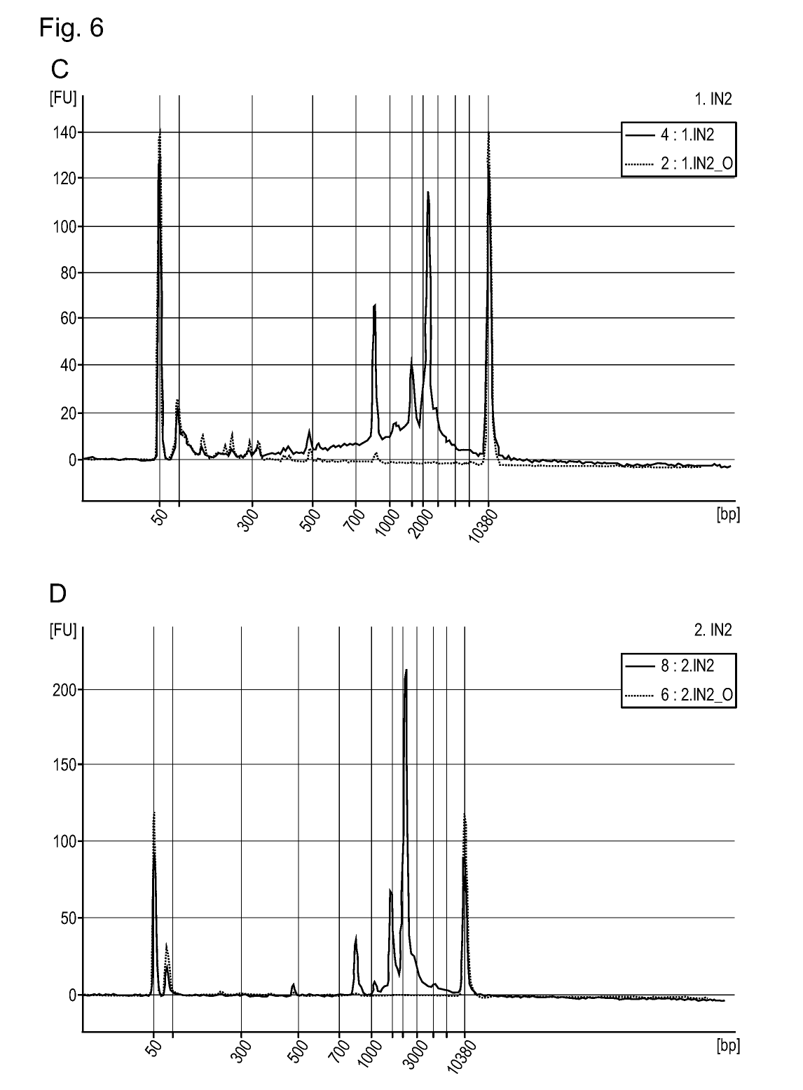

100 ng gDNA from E.coli were processed using the adaptors and primer formulations from the tables above and analyzed on Agilents Bioanalyzer using Agilent DNA chips. FIG. 6 shows the distribution of fragments after amplification with Primer mix 1 (A; C) and Primer mix 2 (B; D).
3: 1.IN1; IN adaptor 1/primer mix 1
1: 1.N1_0; IN adaptor 1/primer mix 1_No template Control
7: 2.IN1; IN adaptor 1/primer mix 2
5: 2.N1_0; IN adaptor 1/primer mix 2_No template Control
4: 1.IN2; IN adaptor 2/primer mix 1
2: 1.N2_0; IN adaptor 2/primer mix 1_No template Control
8: 2.IN2; IN adaptor 2/primer mix 2
6: 2.N2_0; IN adaptor 2/primer mix 2_No template Control

As can be seen the best results were produced by using IN_adaptor 2 and PCR primer mix 1 since a better fragment distribution is achieved.

Further experiments were planned with IN adaptor 2 for optimization of fragmentation.

Different concentrations and incubation temperature as well as purification procedures were tested to obtain a better size distribution of the library and remove remaining adaptor.

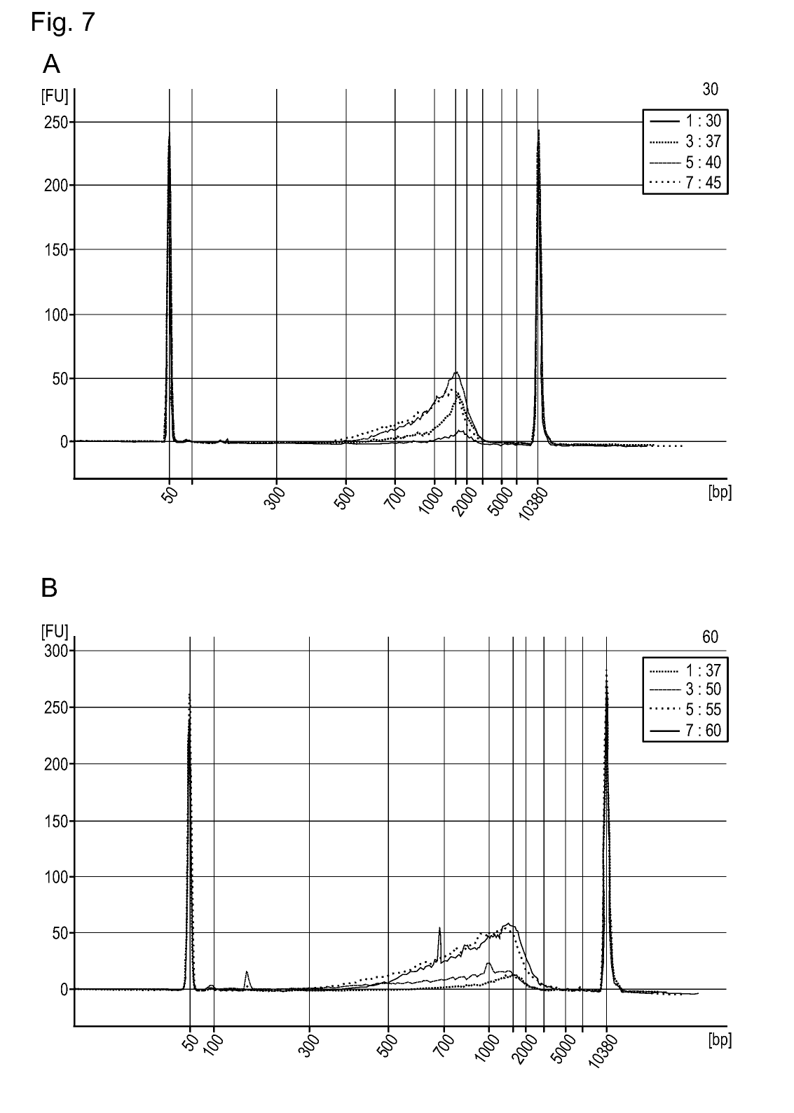

FIG. 7 shows fragmentation and adaptor ligation of 100 ng E. coli gDNA under different incubation temperatures. Surprisingly the in-house HIV-integrase (QHIN_1) used here shows a quite high thermostability which allows incubation of libraries at higher temperatures and results to a better size distribution of the library.

### A:

1:30; incubation of IN adaptor_2 complex with target DNA at 30° C.
3:37; incubation of IN adaptor 2 complex with target DNA at 37° C.
5:40; incubation of IN adaptor 2 complex with target DNA at 40° C.
7:45; incubation of IN adaptor 2 complex with target DNA at 45° C.

### B:

1:37; incubation of IN adaptor 2 complex with target DNA at 37° C.
3:50; incubation of IN adaptor 2 complex with target DNA at 50° C.
5:55; incubation of IN adaptor 2 complex with target DNA at 55° C.
7:60; incubation of IN/adaptor 2 complex with target DNA at 60° C.

According to the presented data the inventors were able to reproduce the plasmid fragmentation results using the HIV-1-integrase enzyme with gDNA. The assay has been optimized to generate a library with a suitable size distribution for several NGS platforms.

Summarizing the above results, the inventors have successfully tested different integrase enzymes to develop the method according to the invention to be used to generate libraries of fragments of tagged target DNA in only one step.

1. A method for generating tagged DNA fragments of a target DNA, comprising
(i) contacting said target DNA with an integrase and at least one DNA adaptor molecule that comprises an integrase recognition site, to obtain a reaction mixture,
(ii) incubating said reaction mixture under conditions wherein a 3′ processing of said at least one adaptor molecule and a strand transfer reaction is catalyzed by said integrase, wherein
(a) said target DNA is fragmented to generate a plurality of target DNA fragments, and
(b) said at least one DNA adaptor molecule is joined to at least one end of each of the plurality of said target DNA fragments,
to generate a plurality of tagged DNA fragments of said target DNA.

2. The method of claim 1, wherein the at least one DNA adaptor molecule is joined to both ends of each of the plurality of said target DNA fragments.

3. The method of claim 1, wherein the at least one DNA adaptor molecule further comprises a site for annealing an oligonucleotide, wherein the site for annealing an oligonucleotide is configured for annealing a PCR and/or sequencing primer.

4. The method of claim 1, further comprising after step (ii) the following step:
(ii)' subjecting said plurality of tagged DNA fragments of said target DNA to a PCR to add to said at least one DNA adaptor molecule a site for annealing an oligonucleotide, wherein said site for annealing an oligonucleotide is configured for annealing a PCR and/or sequencing primer.

5. The method of claim 1, wherein said integrase is selected from the group consisting of: retroviral integrases, HIV integrases, and integrases derived from retroviral integrases.

6. The method of claim 1, wherein said at least one DNA adaptor molecule consists of two nucleic acid molecules comprising complementary nucleotide sequences and being specifically hybridized to each other, selected from the following group:
Adaptor 1 (SEQ ID no. 1+SEQ ID no. 2),
Adaptor 2 (SEQ ID no. 3+SEQ ID no. 2),
Adaptor 3 (SEQ ID no. 6+SEQ ID no. 2),
Adaptor 4 (SEQ ID no. 7+SEQ ID no. 2),
Adaptor 5 (SEQ ID no. 8+SEQ ID no. 4),
Adaptor 6 (SEQ ID no. 9+SEQ ID no. 4),
Adaptor 7 (SEQ ID no. 8+SEQ ID no. 5),
Adaptor 8 (SEQ ID no. 9+SEQ ID no. 5),
Adaptor 9 (SEQ ID no. 14+SEQ ID no. 15),
Adaptor 10 (SEQ ID no. 10+SEQ ID no. 11), and
Adaptor 11 (SEQ ID no. 12+SEQ ID no. 13).

7. The method of claim 1, further comprising after step (ii) and/or (ii)' the following step:
(iii) purifying said plurality of tagged DNA fragments of said target DNA.

8. The method of claim 1, wherein the method is performed within one reaction vessel.

9. A kit for generating tagged DNA fragments of a target DNA, comprising:
(i) an integrase, and
(ii) at least one DNA adaptor molecule comprising an integrase recognition site.

10. The kit of claim further comprising at least one PCR primer pair configured to add in a PCR reaction to said at least one DNA adaptor molecule a site for annealing an oligonucleotide, wherein said site for annealing an oligonucleotide is configured for annealing a PCR and/or sequencing primer.

11. The kit of claim 9, wherein said at least one DNA adaptor molecule further comprises a site for annealing an oligonucleotide, wherein said site for annealing an oligonucleotide is configured for annealing a PCR and/or sequencing primer.

12. The kit of claim 9, characterized in that said integrase is selected from the group consisting of: retroviral integrases, HIV integrases, and integrases derived from retroviral integrases.

13. The kit of claim 9, wherein said at least one DNA adaptor molecule consists of two nucleic acid molecules comprising complementary nucleotide sequences and being specifically hybridized to each other, selected from the following group:
Adaptor 1 (SEQ ID no. 1+SEQ ID no. 2),
Adaptor 2 (SEQ ID no. 3+SEQ ID no. 2),
Adaptor 3 (SEQ ID no. 6+SEQ ID no. 2),
Adaptor 4 (SEQ ID no. 7+SEQ ID no. 2),
Adaptor 5 (SEQ ID no. 8+SEQ ID no. 4),
Adaptor 6 (SEQ ID no. 9+SEQ ID no. 4),
Adaptor 7 (SEQ ID no. 8+SEQ ID no. 5),
Adaptor 8 (SEQ ID no. 9+SEQ ID no. 5),
Adaptor 9 (SEQ ID no. 14+SEQ ID no. 15),
Adaptor 10 (SEQ ID no. 10+SEQ ID no. 11), and
Adaptor 11 (SEQ ID no. 12+SEQ ID no. 13).

14. A method of using an integrase for generating a library of tagged DNA fragments of a target DNA, preferably said library of tagged DNA fragments is a library to be used for DNA sequencing, preferably via next generation sequencing.

15. A nucleic acid molecule comprising a nucleotide sequence selected from the group consisting of SEQ ID NOs. 1 to 15.
